(19) United States
(12) Patent Application Publication
(10) Pub. No.: US 2026/0272466 A1
(43) Pub. Date: Sep. 17, 2026

(54) BLEEDING CONTROL DEVICE

(71) Applicant: William Chase, Avon, CT (US)

(72) Inventor: William Chase, Avon, CT (US)

(21) Appl. No.: 19/563,357

(22) Filed: Mar. 11, 2026

Related U.S. Application Data
(60) Provisional application No. 63/769,858, filed on Mar. 11, 2025.
(60) Provisional application No. 63/813,915, filed on May 29, 2025.

(51) Int. Cl. A61B 17/12; A61B 17/00; A61B 17/34; A61B 90/00; A61M 25/10

(52) U.S. Cl. CPC A61B 17/12136; A61B 17/1204; A61B 17/12099; A61B 17/3423; A61B 17/3462; A61B 2017/00951; A61B 2017/12004; A61B 2017/3492; A61B 2090/037; A61M 25/10186; A61M 2025/1061

(57) ABSTRACT

Bleeding control devices, systems and methods for mitigating bleeding from a wound of a patient. The bleeding control device includes a housing and inflatable balloon. The housing defines a tool opening that allows for insertion of a tool of the bleeding control system from one side of the housing to extend through the housing and beyond a second, opposite side of the housing and into the inflatable balloon. A user of a rod tool or inflation tube tool (or catheter) can manipulate the tool within the inflatable balloon to navigate the inflatable balloon within a wound of a patient before, during and/or after inflation of the inflatable balloon in order to mitigate bleeding of the patient. A strap for use with a bleeding control device that is configured to provide external pressure on an abdomen of the patient while the bleeding control device provides pressure internally within the abdomen.

### CROSS-REFERENCE TO RELATED APPLICATIONS

The present application claims the benefit of U.S. Provisional Patent Application No. 63/769,858, filed Mar. 11, 2025, and the benefit of U.S. Provisional Patent Application No. 63/813,915, filed May 29, 2025, each of which is hereby incorporated by reference in its entirety.

### TECHNICAL FIELD

The present disclosure generally relates to bleeding control at or near the site of an injury at the time when traumatic injury occurs, and more particularly, to an apparatus and method for quickly and efficiently covering and blocking a wound to control bleeding without needing advanced knowledge of first aid procedures.

### BACKGROUND

Current bleeding control techniques for rapid blood loss include applying a tourniquet above a bleeding injury and/or packing a wound with gauze and applying pressure to the wound to reduce bleeding. In either case, if pressure is reduced, even for a short duration, any clots that have formed to slow bleeding may become ruptured and dislodged, causing bleeding to resume at a flow level comparable to the bleeding prior to treatment being applied. Additionally, typical wound packing must be done at a given density, generally immediately following the injury, otherwise the blood flow may not be slowed to an acceptable level and packing is typically not advised for deep penetrating wounds. Moreover, some first aid training must have been acquired for effective use of such prior art techniques, or treatment may not extend the very short duration between untreated bleeding wounds and death. Accordingly, there is a need for a device that can be deployed with minimal effort to assist in such situations and assist in controlling and mitigating bleeding, that is, moreover, portable and easy enough to use in trauma situations by a layperson.

More recently, newer bleeding control devices and methods have been proposed, such as the bleeding control devices and methods disclosed in U.S. Patent Application Publication Number 2023/0190295, which is hereby incorporated by reference in its entirety.

### SUMMARY

A method of controlling bleeding in accordance with the present disclosure includes providing a bleeding control device having a housing that defines a tool opening, and an inflatable balloon held by the housing. The method further includes inserting a tool through the tool opening and into the inflatable balloon, and inflating the inflatable balloon such that the inflatable balloon inflates within a wound of a patient, thereby mitigating bleeding from the wound.

A bleeding control system in accordance with the present disclosure includes a tool and the bleeding control device.

Objects, features and advantages of the present invention will become apparent in light of the description of embodiments and features thereof, as enhanced by the accompanying figures.

### BRIEF DESCRIPTION OF THE DRAWINGS

FIG. 1 is a perspective view of a bleeding control device in a deployed state in accordance with the present disclosure;

FIG. 2 is a front view of the bleeding control device of FIG. 1;

FIG. 3 is a partial, front and cross-sectional view of the bleeding control device of FIG. 1;

FIG. 4A shows various states of a bleeding control device shown from a perspective view in accordance with the present disclosure;

FIG. 4B shows the various states of the bleeding control device of FIG. 4A from a front view in accordance with the present disclosure;

FIG. 5 shows various states of a bleeding control device from a front view in accordance with the present disclosure;

FIG. 6A shows distal end balloon attachment options for a narrow balloon in accordance with the present disclosure;

FIG. 6B shows distal end balloon attachment options for a narrow balloon in accordance with the present disclosure;

FIG. 7A shows distal end balloon attachment option for a wide balloon in accordance with the present disclosure;

FIG. 7B shows distal end balloon attachment option for a wide balloon in accordance with the present disclosure;

FIG. 8 shows rod (or catheter) tip shapes in accordance with the present disclosure;

FIG. 9A shows an asymmetric rod (or catheter) tip shape in accordance with the present disclosure;

FIG. 9B shows rod (or catheter) with a dual material tip in accordance with the present disclosure;

FIG. 10 shows folded balloon options in accordance with the present disclosure;

FIG. 11 shows a balloon running along a length of a rod (or catheter) in accordance with the present disclosure;

FIG. 12 shows a doughnut-shaped balloon housing defining a central opening to allow a rod (or catheter) to insert, thereby unrolling the balloon that is in a rolled-up state in the housing in accordance with the present disclosure;

FIG. 13 shows a balloon attached to a tip of a rod (or catheter) that inflates downward and outward in accordance with the present disclosure;

FIG. 14 shows a balloon attached a distance away from a tip of the rod (or catheter) that inflates outward in accordance with the present disclosure;

FIG. 15 shows a rod (or catheter) inserted through a central opening of a balloon housing having a gasket sealing the area between the housing and the rod in accordance with the present disclosure;

FIG. 16 shows a rod (or catheter) being inserted through a patient wound track and actively steered in accordance with the present disclosure;

FIG. 17 shows a balloon unfurling along a substantial length of a wound track in accordance with the present disclosure;

FIG. 18 shows a rod (or catheter) with a ramped tip in accordance with the present disclosure;

FIG. 19A shows a tip of a rod (or catheter) that expands when inserted beyond external tissue, fat, muscle and peritoneum in accordance with the present disclosure;

FIG. 19B shows a tip of a rod (or catheter) that expands when inserted beyond external tissue, fat, muscle and peritoneum in accordance with the present disclosure;

FIG. 19C shows a tip of a rod (or catheter) that includes an expanding balloon when inserted beyond external tissue, fat, muscle and peritoneum in accordance with the present disclosure;

FIG. 20 shows a long narrow balloon type bleeding control device inserted and inflated within a wound tract of a patient in accordance with the present disclosure;

FIG. 21 shows a variety of balloon types for a bleeding control device in accordance with the present disclosure;

FIG. 22 shows a wide doughnut balloon type bleeding control device inserted and inflated within a patient in accordance with the present disclosure;

FIG. 23 shows a wide pillow shape balloon type bleeding control device inserted and inflated within a patient in accordance with the present disclosure;

FIG. 24 shows a variety of balloon types for a bleeding control device in accordance with the present disclosure;

FIG. 25 shows a multi-balloon bleeding control device in accordance with the present disclosure;

FIG. 26 shows a multi-balloon bleeding control device in accordance with the present disclosure;

FIG. 27 shows a multi-balloon bleeding control device in accordance with the present disclosure;

FIG. 28A shows a variable thickness balloon type bleeding control device in accordance with the present disclosure;

FIG. 28B shows a bleeding control device rod and slidable exterior sheath having an inflatable balloon connected thereto in accordance with the present disclosure;

FIG. 29 shows an inflation path through a catheter of a bleeding control device in accordance with the present disclosure;

FIG. 30 shows an inflation path through a housing of a bleeding control device in accordance with the present disclosure;

FIG. 31A shows a patient with an inflated balloon of a bleeding control device arranged within a wound of the patient, and a strap providing external pressure support in accordance with the present disclosure;

FIG. 31B shows a strap for providing external pressure support for use in conjunction with a bleeding control device in accordance with the present disclosure;

FIG. 31C shows a strap for providing external pressure support for use in conjunction with a bleeding control device in accordance with the present disclosure;

FIG. 32A shows a method of managing a rod (or catheter) of a bleeding control device in accordance with the present disclosure;

FIG. 32B shows a method of managing a rod (or catheter) of a bleeding control device in accordance with the present disclosure;

FIG. 33 shows a method of managing a rod (or catheter) of a bleeding control device in accordance with the present disclosure;

FIG. 34A shows a bleeding control device in accordance with the present disclosure;

FIG. 34B shows the bleeding control device with a balloon in an inflated state and an exposed portion of the rod managed to avoid inadvertent movement in accordance with the present disclosure; and

FIG. 35 shows an insertion force pressure gauge to prevent damage to a patient.

### DETAILED DESCRIPTION

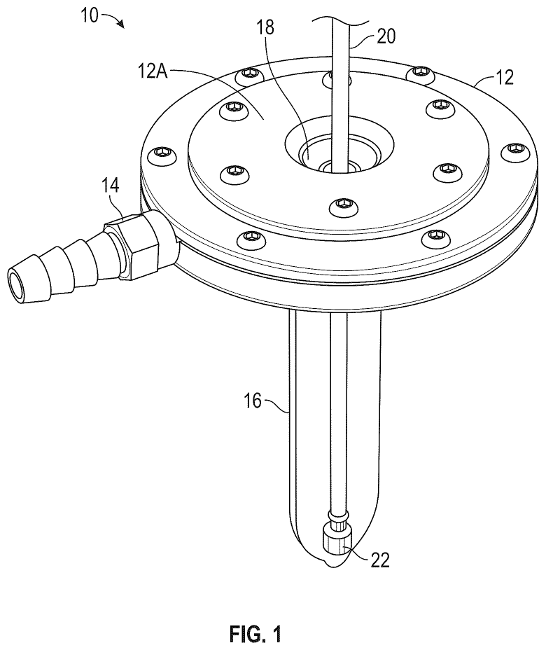

Referring to FIG. 1, a bleeding control device 10 is shown in a deployed state in accordance with the present disclosure. The device 10 comprises a housing 12 having an inflation port 14 and an inflatable balloon 16. The housing 12 defines a tool opening 18 at a first side 12A of the housing 12 that permits a tool 20 to pass therethrough such that a tip 22 of the tool 20 is inserted into the inflatable balloon 16, which is shown in a deployed state (or inflated state).

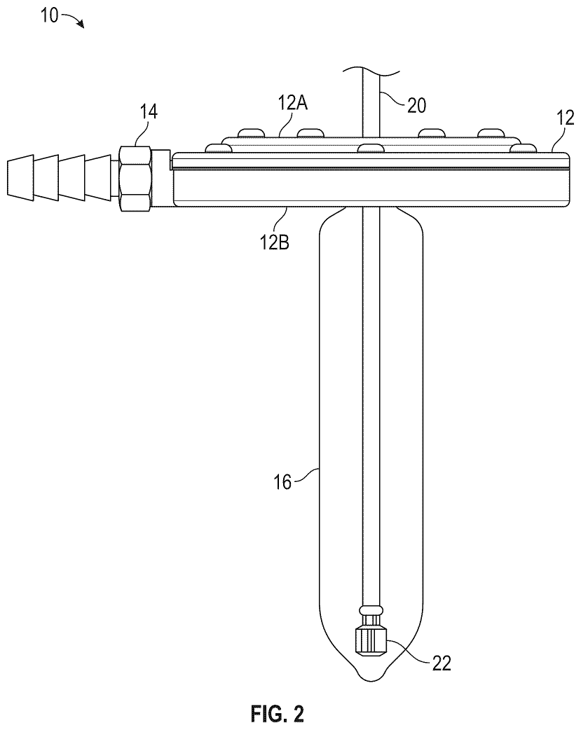

Referring to FIG. 2, a front view of the device 10 shows the tool 20, which in this embodiment is a rod 20, extending through the tool opening 18 from a first side 12A of the housing 12 and extending beyond a second side 12B of the housing 12 that is opposite the first side 12A. The rod 20 is shown with a soft and flexible tip 22.

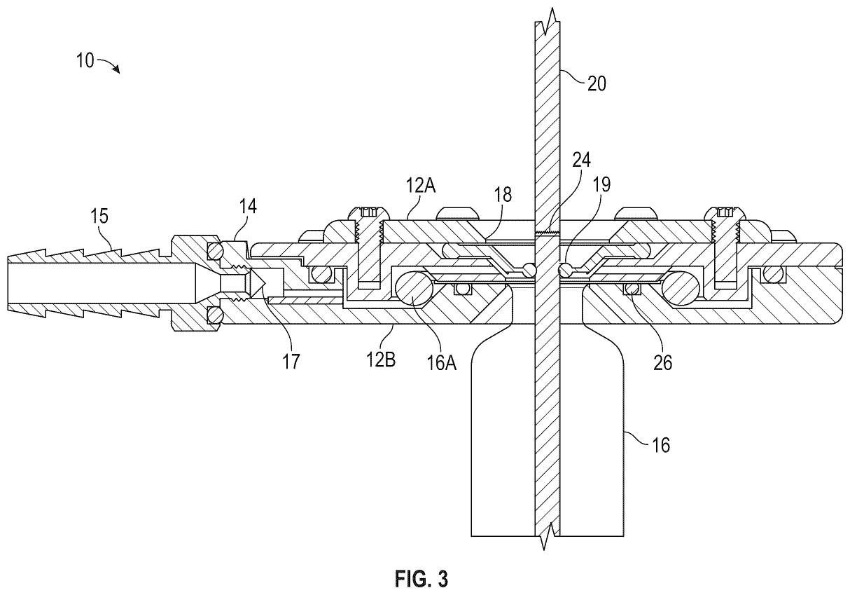

Referring to FIG. 3, a cross-sectional view of the device 10 shows additional elements. The inflatable balloon 16 is held within the housing 12 and includes a rolled or folded end 16A. It is within the scope of the present disclosure for the device 10 to be a “one-time use” configuration where the housing 12 and inflatable balloon 16 are disposed of after use. It is also within the scope of the present disclosure for the housing 12 to be a reusable housing and the inflatable balloon 16 replaced between uses. In such embodiments, the inflatable balloon 16 could be installed in a cartridge-type manner where a portion of the housing 12 engages with the cartridge holding the inflatable balloon 16, or the inflatable balloon 16 attached to the housing 12 in any one or more of known attachment methods. For example and without limitation, the housing 12 could include two or more pieces that clamp together to hold the inflatable balloon 16 therebetween.

The device 10 further comprises a one-way valve 17 in the fluid line between the inflation port 14 and an interior of the inflatable balloon 16. The one-way valve 17 is configured to allow pressure to open the one-way valve 17 to allow fluid (e.g. liquid, gas or some combination thereof) to pass through the valve 17 and ultimately into the inflatable balloon 16. If the pressure from the inflation port 14 decreases below a threshold of the valve 17 or a fluid/pressure source is removed from a fitting 15 connected to the housing 12 and/or inflation port 14, the valve 17 is configured to close and/or seal so that fluid from the inflatable balloon 16 cannot return or exit through the valve 17. Any type of one-way valve 17 is within the scope of the present disclosure, such as, for example and without limitation, ball check valves, flap check valves, pneumatic check valves, non-return valves, duckbill valves, and the like.

In some embodiments, the valve 17 can be omitted from the bleeding control device 10 and the fluid/pressure source can be configured with a one-way valve to prevent the egress of inflation fluid.

The device 10 further includes a sealing element 19 that the tool 20 is configured to extend through when being inserted into the inflatable balloon 16. The sealing element 19 can be a membrane that the tool 20 punctures through when being inserted into the inflatable balloon 16. Alternatively, the sealing element 19 may be any mechanism or sealing means that allows for insertion of the tool 20. In some embodiments, the sealing element 19 is configured for repeated insertion and removal of the tool 20 such that the sealing element 19 continues to seal the interior of the inflatable balloon 16 from the exterior of the device 10 when the tool 20 is removed from the device 10. For example and without limitation, the sealing element 19 can be a sealing gasket (e.g. a duckbill gasket).

In operation, a user arranges the device 10 over a wound of a patient with the second side 12B of the housing 12 facing the wound. The user can then insert the tool 20 (i.e. rod 20) through the tool opening 18 of the housing 12 to navigate the inflatable balloon 16 in the wound of the patient. The shaped tip 22 (FIG. 2) aids in navigating the inflatable balloon 16 while avoiding additional wounds in the patient. Once the user is satisfied with the placement of the inflatable balloon 16 in the wound, the user can cause the inflation fluid to flow into the inflation port 14, thereby causing the inflatable balloon 16 to inflate within the wound and mitigate bleeding. The inflatable balloon 16 can be further manipulated by the tool 20 even after inflation by a user moving the portion of the tool 20 on the first side 12A of the housing 12. In some embodiments, the balloon 16 is sealed to the housing 12 on one or both sides. For example, the tip 22 is outside of the balloon 16 or the balloon 16 is sealed on the housing 12 so the balloon 16 is inflated some distance away from the 12B side of the device.

In some embodiments, one or more attachment elements can be arranged on or at the bottom side 12B of the housing 12 in order to secure the device 10 to a patient. For example and without limitation, an adhesive or sticker can be arranged at the bottom side 12B so that a user can stick or adhere the device 10 to a patient's skin and/or clothing. Or the attachment element can be in the form of a hooked surface or a looped surface of a hook and loop type fastener (such as Velcro®). Advantageously, the attachment elements ensure the device 10 can remain secured in place while medical professionals attend to other tasks when treating the patient. Preferably, the bottom side 12B of the housing 12 includes a substantially flat surface 12B; however, other shapes are within the scope of the present disclosure (e.g. slightly concave shape to the bottom side 12B).

In some embodiments, the tool 20 includes one or more break lines 24 along a length of the tool 20 in a longitudinal direction of the tool 20. The break line(s) 24 is configured to allow a user to fold and/or break the tool 20 at the break line 24. The folding and/or breaking of the tool 20 at the break line 24 allows for all or a portion of the tool 20 above the first side 12A of the housing 12 to be folded down and/or removed entirely, thereby clearing space above the first side 12A of the housing 12 that the tool 20 previously occupied. This clearing of space is a convenience feature as the user does not need to avoid this portion of the tool 20 while administering aid to the patient. This clearing of space also serves as a safety feature by preventing inadvertent contact with the tool 20 that could cause pain and/or harm to the patient. The ability to leave a portion of the tool 20 in the inflatable balloon 16 and fold or remove the other portion of the tool 20 also allows for the patient to be rolled to their opposite side, which may be medically necessary in a variety of circumstances.

The device 10 optionally includes an o-ring seal 26 (FIG. 3), or other type of seal, to prevent inflation fluid from bypassing the interior of the inflatable balloon 16 and escaping the housing 12.

The device 10 may be configured to receive any kind of inflation fluid. For example and without limitation, the inflation fluid may include carbon dioxide, oxygen, nitrogen, argon, and/or water (e.g. saline water), or any combination thereof.

Advantageously, the present disclosure provides bleeding control devices that provide new, optimized and efficient approaches to covering and blocking a wound to control bleeding.

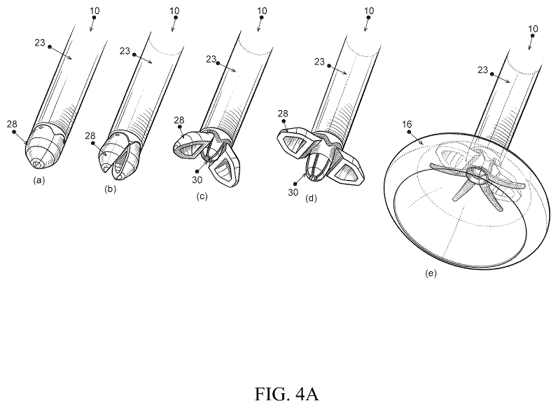

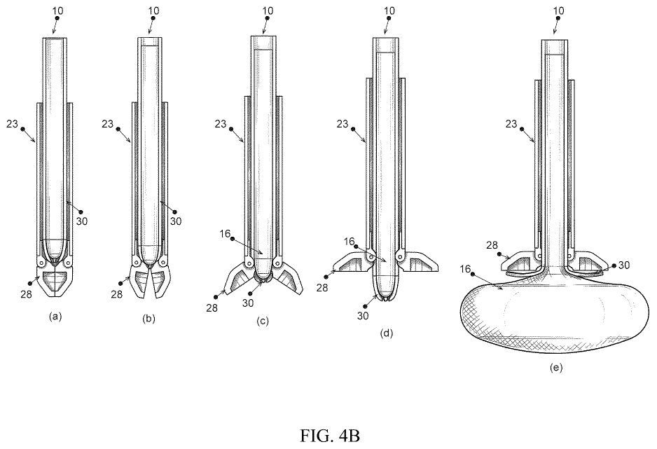

Referring to FIGS. 4A and 4B, a bleeding control device 10 is illustrated in various states as follows: (a) insertion, (b) catheter (or rod) 23 and/or balloon 16 of the bleeding control device 10 opens the tip 28 (the internal sheath 30 is an optional feature that may be included if additional protection of the balloon 16 is desired), (c) balloon 16 emerges (with optional internal sheath 30), (d) the balloon 16 is positioned at proper depth in abdominal cavity of patient, and (e) balloon 16 is inflated.

In some embodiments, a rod with a sensor(s) (e.g. a camera, bleeding detection probe, etc.) is inserted through the opened tip 28 prior to insertion or inflation of the balloon 16. Alternatively, the tip 28 includes or is embedded with the sensor(s). This allows a user to survey the wound and/or internal organs, tissue, blood vessels of the patient prior to mitigation with an inflated balloon(s). The tip 28 may be utilized to move fascia within the patient prior to deployment of the inflatable balloon 16.

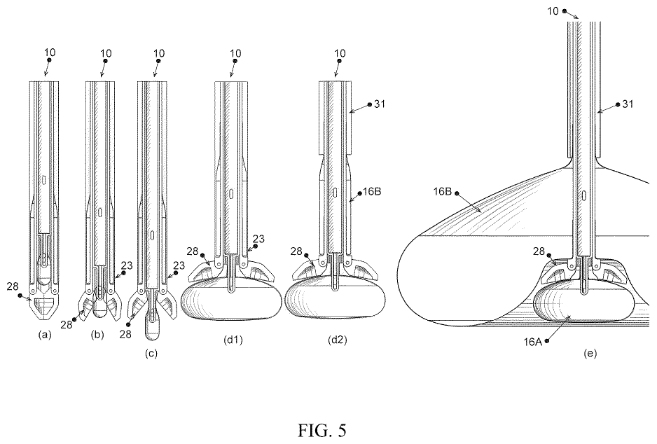

Referring to FIG. 5, a bleeding control device 10 is shown in various states as follows: (a) insertion, (b) internal balloon 16A and/or catheter 23 open the tip 28, (c) the internal balloon 16A is positioned at the proper depth, (d₁) the internal balloon 16A is inflated, (d₂) the optional external sheath 31 is removed (if equipped), thereby exposing the external balloon, (e) the external balloon (large balloon) is inflated for bleeding mitigation.

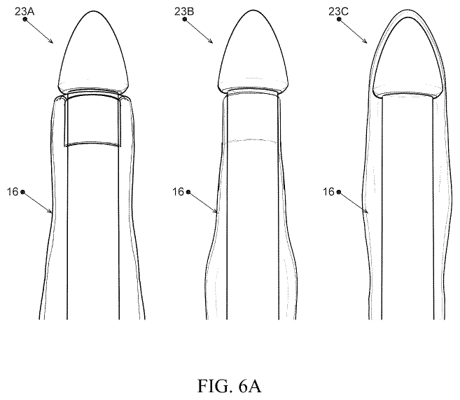

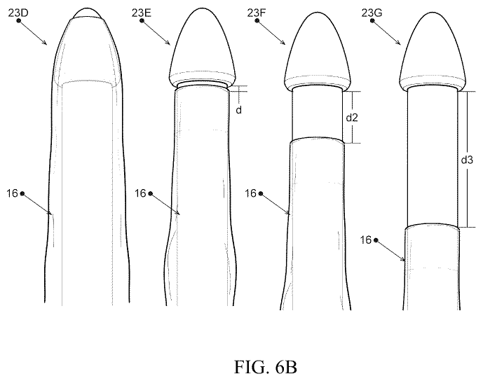

Referring to FIG. 6A, narrow long balloon attachment options are shown for attachment to a distal end of a rod (or catheter) 23. The balloon attachment options include dual neck inverted 23A and dual neck traditional 23B (the left and central embodiments shown in the figure, respectively) and dip molded/welded 23C that encapsulates the tip of the rod (right embodiment shown in the figure). In each of the shown embodiments, the balloon 16 extends beyond the tip of the rod 23 when the balloon 16 is inflated.

Referring to FIG. 6B, additional embodiments for narrow long balloon attachment options are shown. The shown embodiments in this figure are of a dual neck attachment configuration where the distance from the tip is increased successively from the embodiment 23D (no distance, the balloon 16 is welded or otherwise attached to the tip; though some portion of the tip may be exposed as shown in the circle area) to the next embodiment 23E having a short distance d₁ of the balloon attachment to the tip, to the next embodiment 23F (second from the right) having a medium distance d₂, to the next embodiment 23G (right embodiment shown in the figure) having a long distance d₃ from the tip to the balloon attachment point end. The further the balloon 16 is attached from the tip of the rod 23 may provide protection of the balloon 16 from damage during insertion or better inflation characteristics.

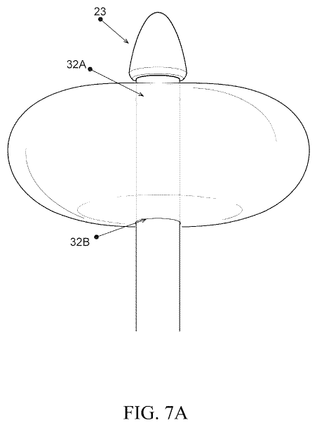

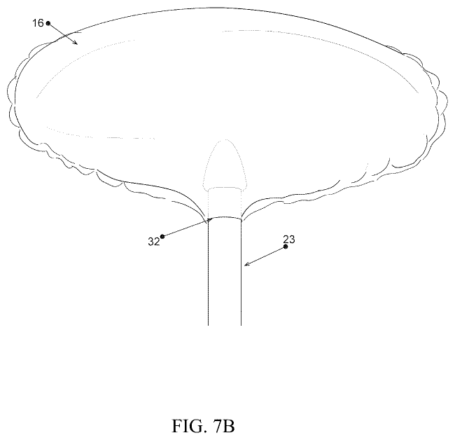

Referring to FIGS. 7A and 7B, wide balloon attachment options are shown for attachment to a distal end of a rod (or catheter) in accordance with the present disclosure. The balloon attachment include dual neck (e.g. for a doughnut shape embodiment as shown in FIG. 7A) where the balloon attaches at two points 32A, 32B on the rod 23. The attachment options also include single neck, where the balloon 16 attaches at a single point 32 (e.g. for a pillow shape embodiment as shown), as shown in FIG. 7B. A “point” of connection may be a continuous point along a circumference of the rod at a particular axial position of the rod, or may be a plurality of scattered points along a circumference of the rod at a particular axial position of the rod.

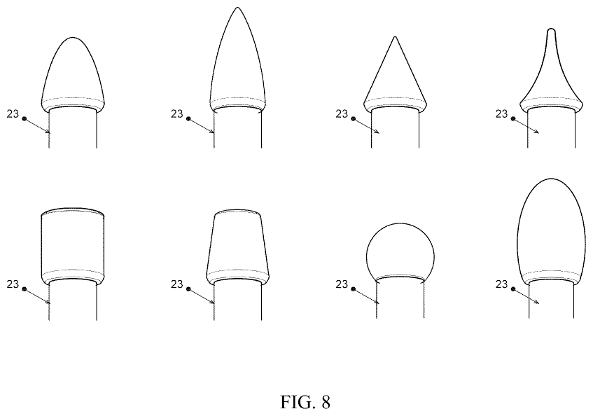

Referring to FIG. 8, various rod (or catheter) 23 tip shapes are shown in accordance with the present disclosure. The tip shapes include: conic, cone, tapered cone, spherical, flat, and others. The relative length and diameter of the tips are configurable as desired. These varieties of tip shapes and relative sizes and shapes are non-exhaustive and other tip shapes, sizes and designs are within the scope of the present disclosure.

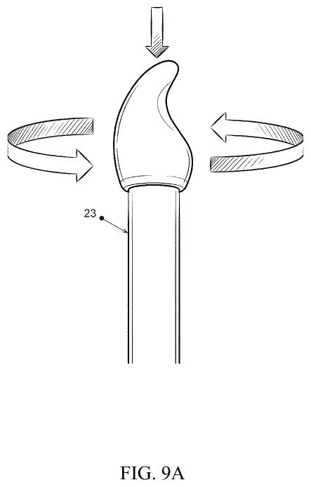

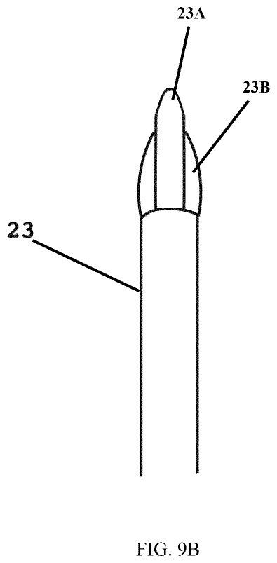

Referring to FIG. 9A, an asymmetric tip for a rod (or catheter) 23 is shown in accordance with the present disclosure. In operation, as the user rotates the rod (or catheter) 23 the asymmetric tip may be more likely to find the wound tract path or give feedback to the user, which will aid in positioning the rod and/or balloon in the desired depth and location more quickly and easily.

In some embodiments, the tip of the rod (or catheter) is configured to be inflated with air and/or other fluid. In operation, the tip can be inflated to be relatively rigid during insertion, and then deflated after insertion (i.e. relatively soft, or at least softer than the rigid firmness during insertion) to blunt the tip. When the tip is inflated to a desired pressure, the forces acting on the inflated tip may serve to provide feedback as the tip deforms during navigation of the rod (or catheter) in a wound of a patient. In operation, the pressure in the inflatable tip may be relieved once it moves through the peritoneum. Alternatively, the tip may be rigid initially and after through the fascia inflated to increase the surface area of the tip.

In some embodiments the tip is configured to be expandable allowing for a longer size during initial entry and navigation and expansion when inside the abdominal cavity of the patient. The expansion of the tip may serve to create a void (i.e. make room) and the tip may be configured to be manually retracted in some embodiments.

As shown in FIG. 9B, in some embodiments the tip is configured as a dual material tip where there is a soft material core 23A and firm material outer shell 23B, i.e. the tip core is a softer material than the outer surface of the tip. This results in the inner core 23A being compressed so it does not extend beyond the outer core 23B material distally.

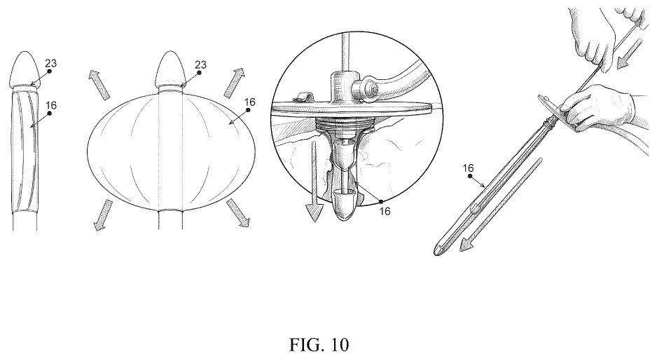

Referring to FIG. 10, folded balloon options are shown to allow a bleeding control device to achieve a more compact profile during insertion in accordance with the present disclosure. The balloon may be folded by pleating, rolled up, twisted, origami folding, accordion, and other folding techniques to reduce the overall footprint of the balloon and/or device in order to make insertion of the device easier. The folding allows the balloon to more easily fit inside the wound opening before it is inflated.

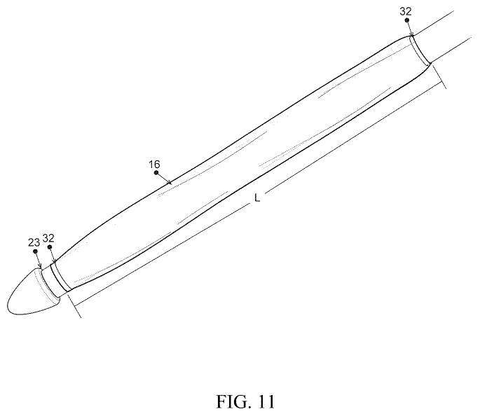

Referring to FIG. 11, a bleeding control device embodiment is shown in accordance with the present disclosure. The embodiment includes a balloon 16 that is long and narrow that runs along a length L of the rod (or catheter) 23. The balloon 16 is attached at two points 32 (both ends of the balloon). Many lengths are within the scope of the present disclosure. In some embodiments, the balloon 16 is attached to a tube that freely slides along the insertion rod (the inter rod with the tip), and include a fluid connection separate from the tube.

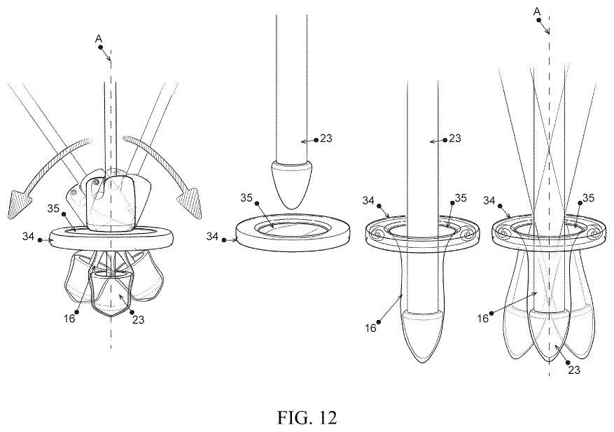

Referring to FIG. 12 a bleeding control device 10 is where the balloon 16 unrolls from a rolled-up state as the rod (or catheter) 23 is inserted into a wound tract of a patient. The balloon 16 is captured or arranged within a disc 34 or other housing as the rod 23 pushes through a central opening 35 defined by the disc (or housing) 34. As the rod 23 advances further into the wound of the patient, the balloon 16 is unrolled within the housing and extends with the rod 23 into the wound of the patient. The rod 23 is able to rotate and/or pivot with respect to a longitudinal axis A of the rod 23 during insertion in order for a user to more easily navigate a wound track (e.g. puncture wound) of a patient. In some embodiments, the balloon 16 is captured in a doughnut shaped package with only a small portion exposed with the tip for insertion.

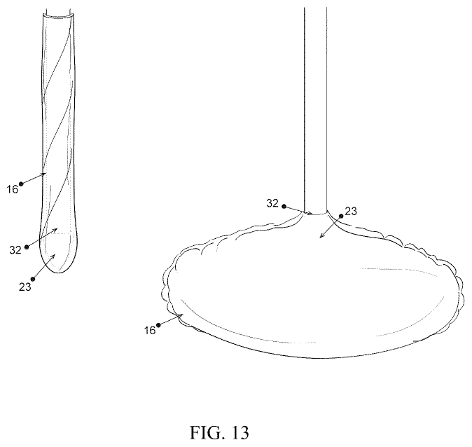

Referring to FIG. 13, the balloon 16 is attached (or welded) to a tip of the rod (or catheter) 23 and then folded along a length of the rod 23 (left view of FIG. 13). The balloon 16 is folded prior to insertion or inflation of the balloon 16 within a patient wound. When inflated, the configuration of the balloon 16 and/or the attachment point 32 cause the balloon 16 to inflate downward and outward as shown in the right view of FIG. 13.

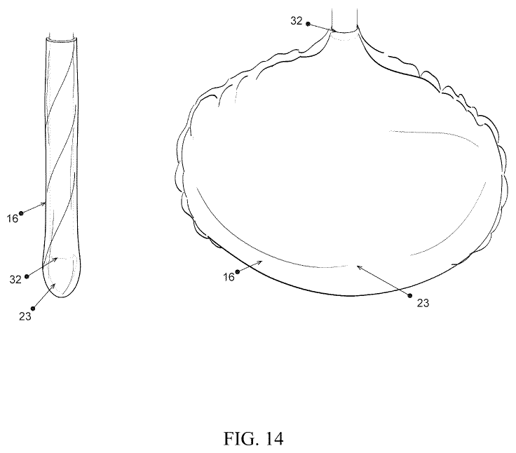

Referring to FIG. 14, the balloon 16 is attached (or welded) at a length up from the tip of the rod (or catheter) 23 and folded tight around the length of the rod 23 below the attachment point (left view of FIG. 11). When inflated, the configuration of the balloon and/or the attachment point cause the balloon to inflate outward as shown in the right view of FIG. 14. This may incorporate a breakaway weld or seam to encourage portions of the balloon to inflate before others.

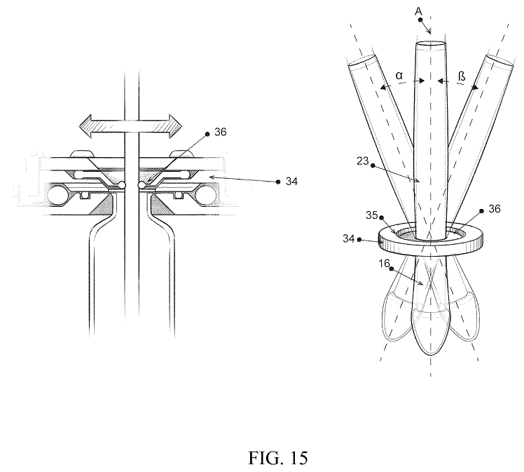

Referring to FIG. 15, a rod (or catheter) 23 is inserted through a balloon housing 34 having a gasket 36 that seals the area between the housing 34 and the rod 23 while the rod 23 is inserted through the balloon housing 34. In some embodiments, the gasket 36 will self-seal and seal the central opening 35 when the rod 23 is removed from the balloon housing 34. The central opening 35 is sized and/or shaped to allow for the rod 23 to rotate and/or bend at angles α, β with respect to a longitudinal axis A of the rod 23 and with flexibility built into the gasket 36 to allow such movements without breaking the seal between the gasket 36 and the rod 23. The range of motion provides freedom for the user to manipulate rod 23 as desired to navigate the wound of the patient.

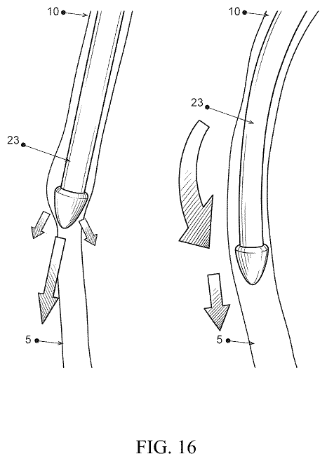

Referring to FIG. 16, a rod (or catheter) of a bleeding control device 10 being steered through a patient wound 5 is shown. The rod 23 may be configured as a rigid material that allows to easily push the rod 23 through the exterior tissue, muscle, fat, and preatrium into the abdominal cavity. In some embodiments, the rod 23 may be flexible but with enough rigidity to stay on path within the wound tract 5 without disrupting or causing damage as the rod is steered into the abdominal cavity of the patient. In some embodiments, the rod is configured to have the relative rigidity of the rod dynamically altered by a user as desired. For example, the rod may be inflated, include piezo electric elements, or include a guide wire/lumen.

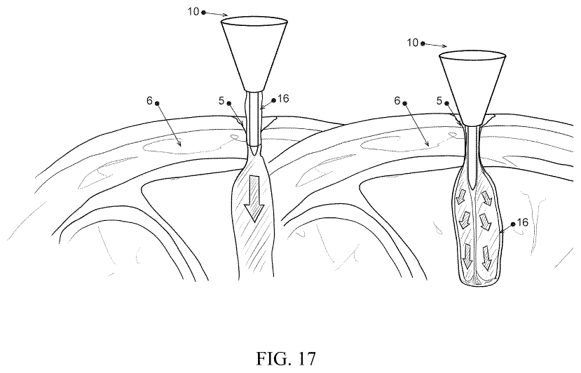

Referring to FIG. 17, a balloon unfurling through a wound tract 5 is shown. The rod (or catheter) 23 locates the device 10 on the entry of the wound 5, but does not progress along the entirety of the wound tract 5 or even a substantial portion of the wound tract 5. Instead, the balloon 16 inflates and thereby unrolls and/or unfurls along a substantial length of the wound track 5, e.g. unrolls and/or unfurls along a majority of the length of the wound track 5. The rod 23 may be configured to be relatively short and, thus, not capable of being inserted throughout the majority of the length of the wound track 5 because the balloon 16 is arranged to unroll and/or unfurl throughout the wound 5 upon inflation.

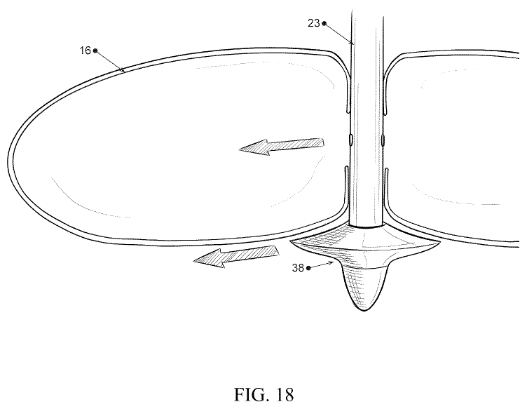

Referring to FIG. 18, a rod (or catheter) 23 is shown with a ramped tip 38 that is sized and/or shaped to help direct the inflatable balloon 16 to inflate outwards (i.e. in a radial direction with respect to a longitudinal or axial length of the rod 23). This support of inflation in a particular direction may be to direct the balloon 16 to inflate into the void between organs and external tissue and peritoneum. In some embodiments, the ramped tip 38 is flexible.

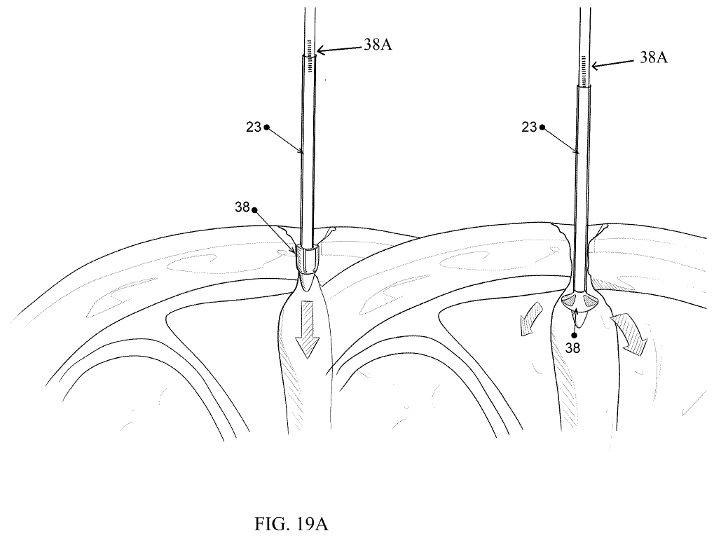

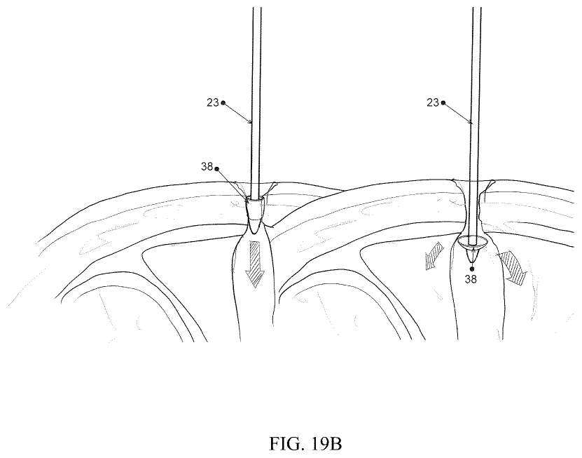

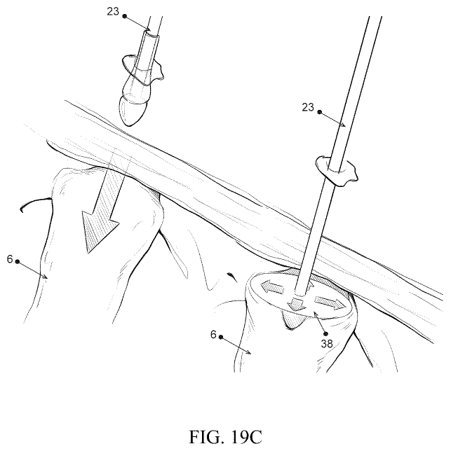

Referring to FIG. 19A, a rod (or catheter) 23 is shown with a tip 38 that is configured to provide feedback to a user in order to indicate to the user when the tip 38 has progressed through the external tissue, fat, muscle, and peritoneum. The feedback may be haptic due to resistance to pushing the rod 23 being reduced when the tip 38 expands and/or wings at or near the tip 38 expand. In some embodiments, there may be an indicator 38A on the proximal end of the rod 23 that indicates to a user the state of the tip 38 at the distal end as the indicator becomes revealed by moving relative to an outer tube of the rod 23, e.g. a visual and/or auditory indicator that indicates when the tip 38 or the wings expand. This tip 38 may be under tension to extend when it is past denser tissue or it may be manually moved by a connection controlled by the user. The control may include markings or scale to represent how wide the tip 38 has expanded.

Referring to FIG. 19B, a tip 38 is shown that makes a click and/or pop sound, and/or generates haptic feedback when the tip 38 progresses through the external tissue, fat, muscle, and peritoneum.

Referring to FIG. 19C, a tip 38 is shown that provides feedback so that the user knows when the tip 38 has progressed through the external tissue, fat, muscle, and peritoneum. A small inflatable balloon 16 (doughnut-shaped in this embodiment, but other shapes are within the scope of the present disclosure) that inflates to maintain the rod (or catheter) 23 in the abdominal cavity 6, thereby preventing inadvertent withdrawal of the rod 23 should the patient unexpectedly move or the proximal end of the rod 23 be inadvertently touched or moved.

In some embodiments, the expanding tip 38 is user actuated to expand and/or retract (e.g. when a string or rod is pushed or pulled) to allow the user to probe the patient wound and determine if the tip is at a desired position within the wound or abdominal cavity.

The expanding tip 38 of a rod (or catheter) 23 may also serve to be used to pull the flesh of the patient back to generate a space for the balloon of the bleeding control device to inflate. Once inflated, the device can be pushed down to provide pressure on the bleeding vessel(s).

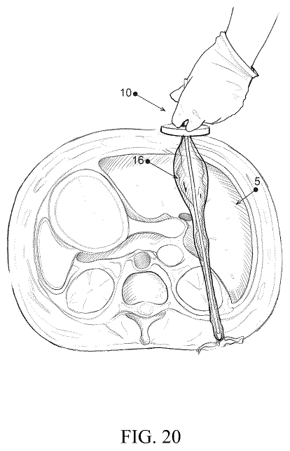

Referring to FIG. 20, a long narrow balloon 16 of a bleeding control device 10 is shown that is configured to expand and apply even pressure along the wound tract 5. In some embodiments, low pressure may be provided to the balloon 16 with a geometry (i.e. size and/or shape) that is larger than the expected wound geometry (i.e. size and/or shape) of the wound tract 5.

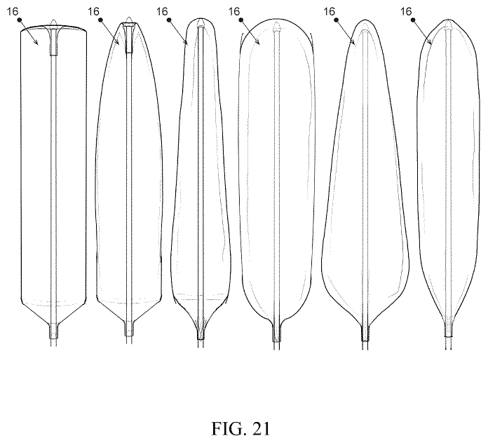

Referring to FIG. 21, a variety of balloon 16 shapes for a bleeding control device 10 are shown. The balloon 16 shape may be pill shape, tapered, cylindrical, and/or having wall-thickness changes. These shapes are non-limiting and non-exhaustive as other shapes are within the scope of the present disclosure. The balloon 16 shape does not need to be symmetrical and can be configured as generally any suitable shape for occupying various wound shape/paths.

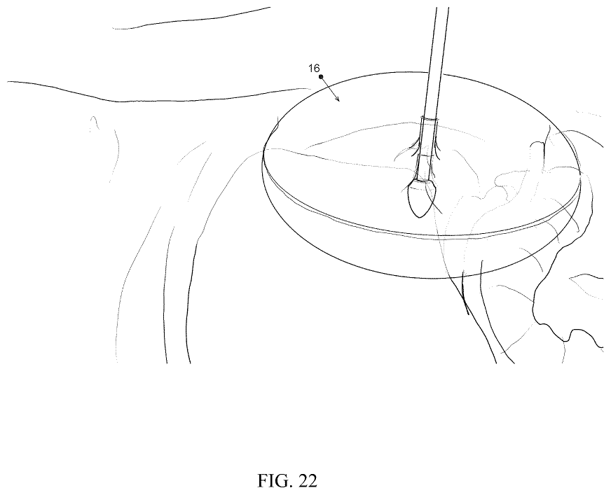

Referring to FIG. 22, a wide doughnut shape balloon 16 of a bleeding control device 10 is shown that is configured to expand outward to apply even compressive pressure across organs in the abdominal cavity. In some embodiments, low pressure may be provided to the balloon 16 and the material expands outward between the organs and exterior tissues of the patient.

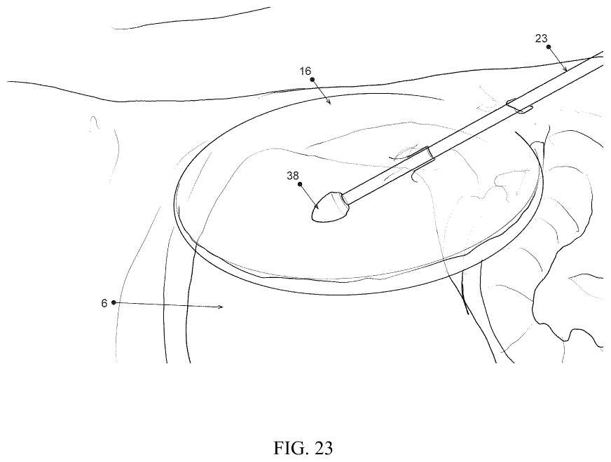

Referring to FIG. 23, a wide pillow shape balloon 16 of a bleeding control device 10 is shown that is configured to expand outward from the tip 38 of the rod (or catheter) 23 to apply even compressive pressure across the organs in the abdominal cavity 6. In some embodiments, low pressure may be provided and the material expands outward between the organs and exterior tissues of the patient.

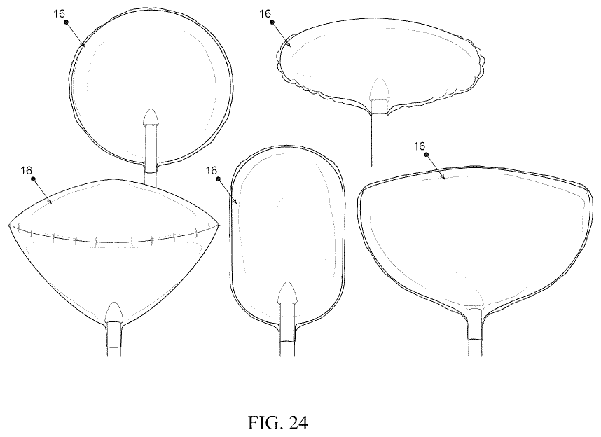

Referring to FIG. 24 a variety of balloon 16 shapes for a bleeding control device are shown for wide pillow shape balloon types. The balloon 16 shape may be pill shape, circular/disc shape, kidney shape, wedge shape, and triangular shape. These shapes are non-limiting and non-exhaustive as other shapes are within the scope of the present disclosure. In some embodiments, the balloon 16 shape is arranged to inflate into a curved or cupped shape of a wound.

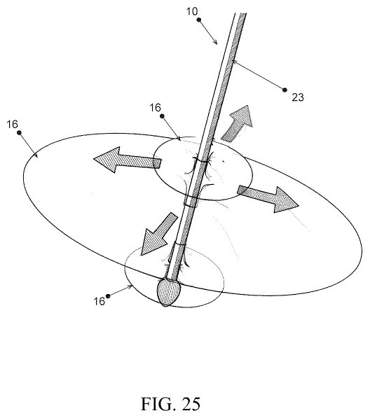

Referring to FIG. 25, a multi-balloon bleeding control device 10 is shown. The two or more balloons 16 are arranged on the rod 23 so that the balloons can be made of different sizes and inflated independently. This assists in deploying the balloons 16 in a way to maximize the placement and deployment.

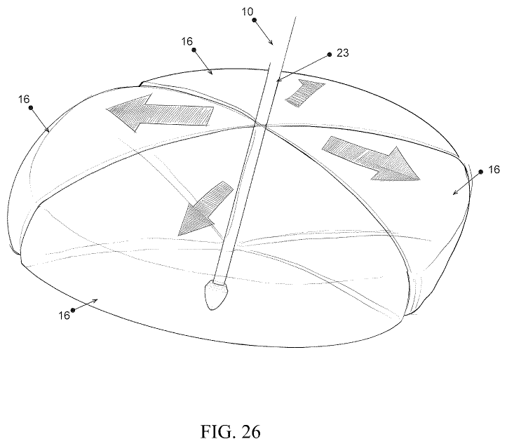

Referring to FIG. 26, a multi-balloon bleeding control device 10 is shown. An array of multiple balloons anchored to the rod (or catheter) that expand outward to cover a large surface area. The balloons 16 may be configured as similar or different materials or stiffness. The geometry of the balloons 16 may be arranged to provide tight nesting, i.e. little to no air space between the balloons 16. Each balloon 16 may be configured to inflate independently, or all could inflate together like a web.

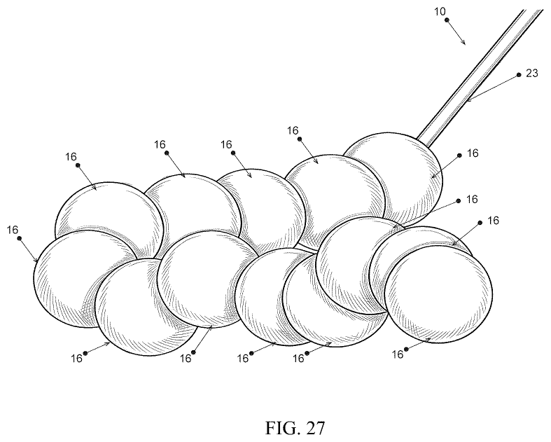

Referring to FIG. 27, a multi-balloon bleeding control device 10 is shown. A chain (or string) of balloons 16 is arranged to expand and self-locate in the abdominal cavity by finding void space to inflate within. By having the balloons 16 fill void space, the chain of balloons 16 maintains an even compression of the abdomen to stop bleeding. The linked chain of balloons 16 enables expansion of the balloons 16 to position themselves in unique orientations/configurations that advantageously are suited for atypical wound shapes that a standard balloon type may not be able to effectively address. In some embodiments, the device 10 includes an optional mechanism for rapid inflation and deflation of the chain of balloons 16. The mechanism may be a simple oscillating valve or reciprocating pneumatic circuit. This would inflate and deflate the balloons 16 in order to cause the balloons 16 to self-position into the greatest void.

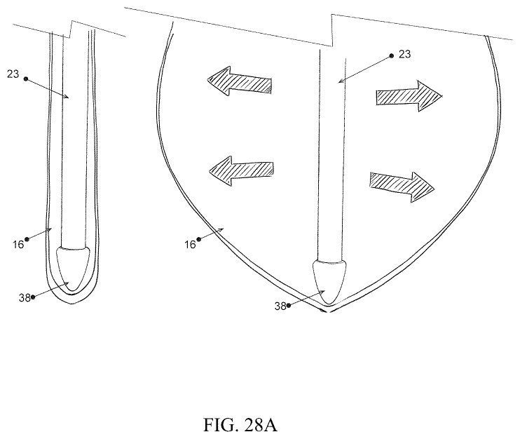

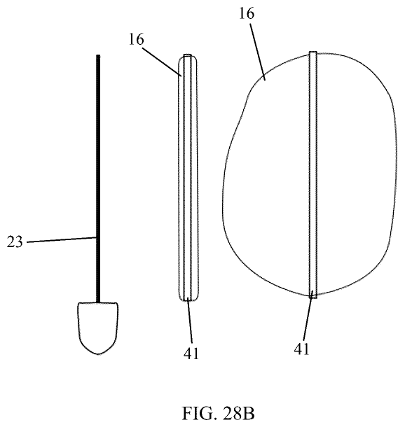

Referring to FIG. 28A, a variable thickness balloon type of a bleeding control device is shown. The balloon material thickness is thicker near where the tip 38 of the rod (or catheter) 23 navigates the balloon, i.e. where the tip 38 of the rod 23 pushes on the interior of the balloon 16, in comparison to other parts of the balloon 16. This wall thickness variation serves to enable a desired inflation of the balloon 16 as well as provide for increased durability of the balloon while navigating the balloon 16 within a wound of a patient.

In some embodiments, the balloon 16 is attached to an exterior sheath 41 that rides on an interior rod (or catheter) 23. In operation, the rod 23 is first inserted into the abdominal cavity/wound tract. The balloon 16 on a sealed exterior sheath 41 is then slid down the rod 23 into the abdominal cavity/wound tract. Upon inflation of the balloon 16, the balloon 16 would self-align itself on the rod 23 as to expand where the void is found in the cavity. In some embodiments the inverse configuration is provided, and the sheath 41 controls the inflation portion.

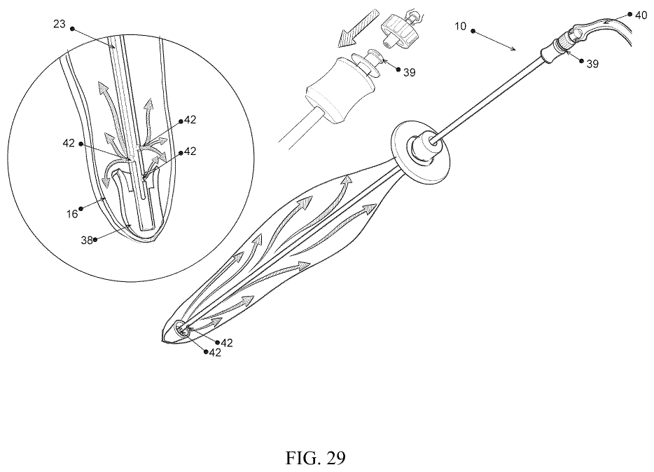

Referring to FIG. 29, an inflation path of a bleeding control device 10 is shown. The inflation medium is provided to the balloon 16 through the catheter. A proximal end 39 of the catheter 23 is arranged to be fluidly connected with a fluid source 40. The catheter 23 is shown with a particular number of inflation ports 42 at or near the distal end that fluidly connect the interior of the catheter 23 with the interior of the balloon 16. Any number of inflation ports 42 and virtually at any position along catheter 23 is within the scope of the present disclosure.

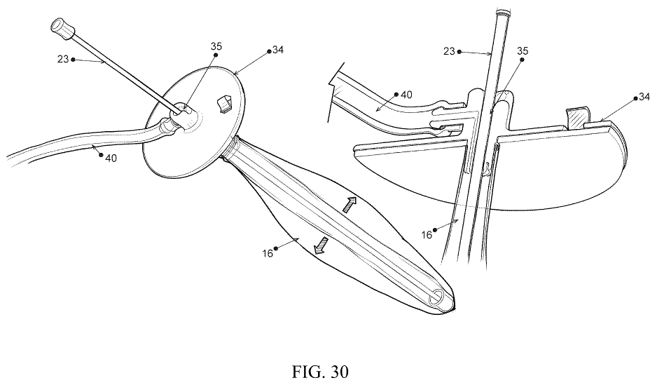

Referring to FIG. 30, an inflation path of a bleeding control device is shown. The inflation medium inflates the inflatable balloon 16 through a secondary component, a housing 34. A proximal end of the balloon 16 is attached (or welded) to the housing 34 or the proximal end of the balloon 16 is contained in the housing 34 preventing the inflation medium from leaking. A rod 23 is inserted through a central opening 35 of the housing 34 and navigates the balloon 16 into a wound of a patient. A tubing 40 is provided to a proximal side of the housing to provide the inflation fluid to the housing 34, that then provides the inflation fluid to the inflatable balloon 16.

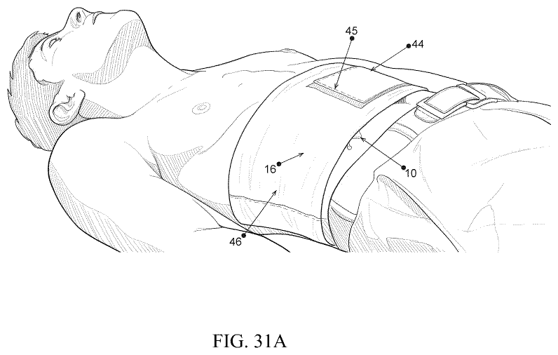

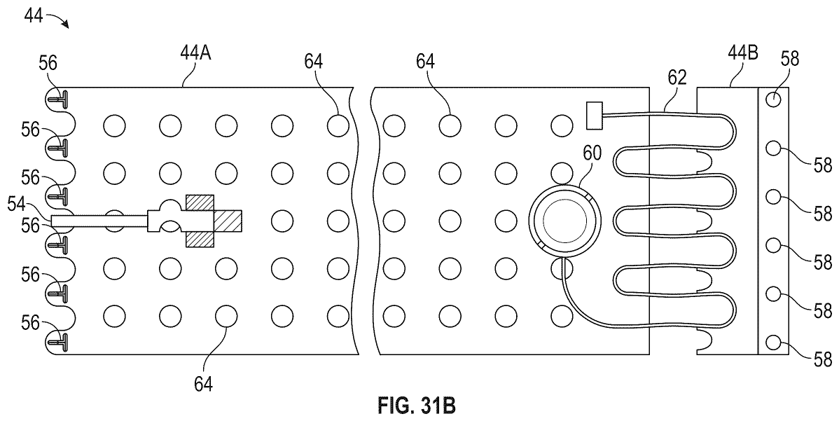

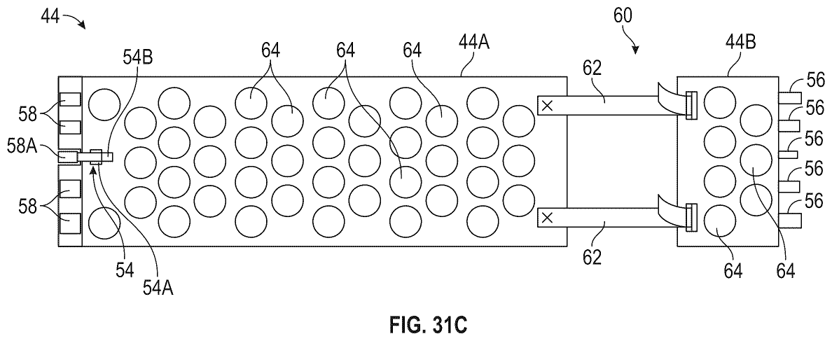

Referring to FIG. 31A, a strap (or retention device) 44 is shown attached around the abdomen of a patient that has an inflated balloon of a bleeding control device 10 arranged within a patient to control bleeding of the patient. The strap 44 is provided in accordance with methods of the present disclosure to maintain an external pressure on the bleeding control device 10 and the patient/victim to maintain the balloon 16 in the desired position and applying the desired pressure to keep the balloon 16 from moving, thereby supporting the stopping of the bleeding. The strap 44 is configured to be tensioned as desired to provide the desired amount of external pressure on a bleeding control device 10. For example and without limitation, an end 45 of the strap 44 may be configured to secure at a plurality of different positions along an intermediate portion 46 of the strap 44 to obtain a desired amount of tension or external pressure on the bleeding control device 10. The end 44 may be configured with hook and/or loop type fastener elements, adhesive, or other securing/attachment mechanisms.

Referring to FIG. 31B, a strap (or retention device) 44 is shown. The strap 44 includes a force gauge (or strain gauge) 54. One end of the strap 44 includes one or more attachment elements 56 that are configured to attach or secure to one or more corresponding or complementary attachment elements 58 at the opposite end of the strap 44 in the longitudinal direction of the strap 44. The attachment elements 56, 58 are shown as hooks 56 configured to be retained by openings 58, however, any attachment element or mechanism is within the scope of the present disclosure. For example and without limitation, the attachment elements may be components of a double sided buckle mechanism, like the kind used on backpacks.

Once the attachment elements 56, 58 are attached or secured together, a user can cause the strap 44 to tension by adjusting a tension adjuster 60, which causes one or more tensioning elements 62 to pull the two portions 44A, 44B of the strap 44 together. The force gauge 54 may be configured to indicate when the strap 44 is at or within a predetermined level of tension, for example, by showing a green color. The force gauge 54 may also be configured to indicate when the strap 44 is at or within an unacceptable level of tension (e.g. not enough tension or too much tension) by indicating, for example, a red color. Alternatively, the force gauge 54 could provide numerical or symbolic indications tension instead of color-coded indications.

Advantageously, the strap 44 when tensioned provides sufficient application of pressure about the circumference of the patient's abdomen to provide an external force on the wound and/or the patient's abdomen generally. While a bleeding control device is providing pressure from within the wound of the patient, the strap 44 can provide an external pressure to prevent undesirable expansion of the wound from within by the bleeding control device, and to also provide desired pressure on the bleeding portions of the wound to mitigate bleeding.

The strap 44 body defines a plurality of openings (or holes) 64. The openings 64 are large enough to permit a bleeding control device 10 to pass therethrough. There being a plurality of openings 64 arranged along the length and/or height of the strap 44 advantageously permits the strap 44 to be applied to many different body types and sizes and still make an opening 64 available for passing through a bleeding control device 10 when the strap is applied to a patient. The strap 44 body may be made from neoprene and/or nylon, however other strap 44 body materials are within the scope of the present disclosure. The strap 44 body may also include one or more areas of hook and/or loop fastener components to attach tools and other equipment to the strap 44.

Referring to FIG. 31C, a strap (or retention device) 44 is shown. The strap 44 is similar to the embodiment of FIG. 31B in the sense that the strap 44 includes a force gauge 54, a tension adjuster 60 for adjusting the tensions between two portions 44A, 44B, and a plurality of openings 64. However, in the embodiment shown in FIG. 31C, the tension adjuster 60 comprises two straps 62 fed through openings in the portion 44B. The straps 62 can have any attachment mechanism for adhering to itself such as, for example, hook and loop type fasteners, magnets, adhesives, etc. While two straps 62 are shown, any number of straps 62 (including only one strap 62) are within the scope of the present disclosure.

The force gauge (or strain gauge) 54 includes a colored piece of fabric or other material 54A (e.g. green) that becomes visible through a window 54B of the force gauge 54. The material 54A may be multi-colored such as red and green so that when the portions 44A, 44B are at a predetermined level of tension (or within a predetermined tension range) the green colored material 54A is visible through the window 54B and when the tension is greater than the predetermined tension level or range, the red colored material 54A becomes visible through the window 54B.

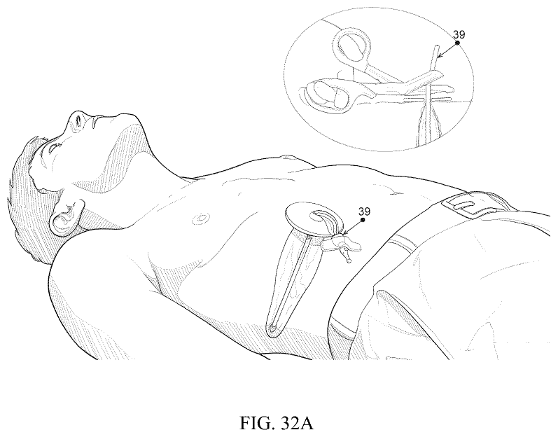

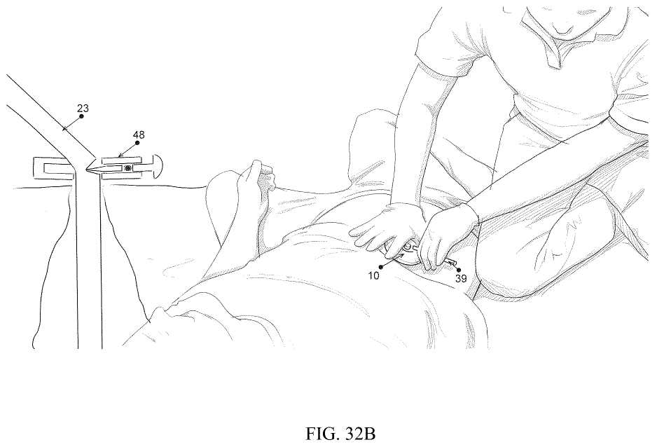

The attachment elements 56, 58 in the FIG. 31C embodiment are switched with respect to the FIG. 31B embodiment in the sense that the hooks 56 are arranged on the right side portion 44B (from the perspective of FIG. 31C) and the openings 58 are arranged on the left side portion 44A. The opening 58A is the opening that the force gauge 54 is connected to sensing the current level of tension of the strap 44. Referring to FIG. 32A, a method of managing an exposed portion of a rod (or catheter) 23 is shown. To minimize the likelihood of the rod 23 causing damage and/or pain to the patient, a proximal exposed portion 39 of the rod 23 may be managed to prevent inadvertent movement of the rod 23. In some embodiments, the exposed portion 39 of the rod 23 is clipped (or cut) and removed from the device (upper right view of FIG. 32A). In some embodiments, the rod 23 can be bent flat so that the exposed portion 39 of the rod (or a majority of the exposed portion) is bent towards the patient, thereby reducing the likelihood of inadvertent contact. The rod 23 can be bent after cutting of the rod 23.

Referring to FIG. 32B, a method of managing an exposed rod (or catheter) is shown. In some embodiments, the cutting implement 48 is integrated into the bleeding control device 10 and, thus, separate scissors or other cutting implement (shown in FIG. 32A) is not necessary. The cutting device 48 shown in FIG. 32B surrounds the rod 23 and includes a manually actuated mechanism that includes an interior cutting blade that can be configured to cut the rod 23 completely for removal of the proximal portion 39 to the cutting point, or alternatively cut the rod 23 to a partial degree to allow for bending of the rod 23 as discussed above. This may also include a clamping mechanism to hold the retained portion of the rod in place.

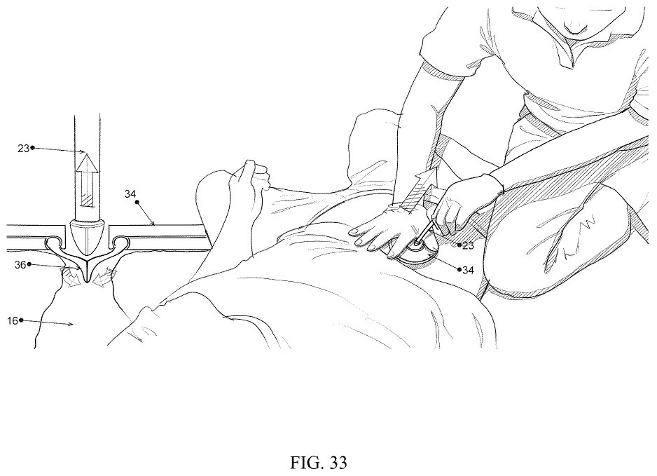

Referring to FIG. 33, a method of removing a rod (or catheter) 23 of a bleeding control device 10 is shown. Instead of leaving the rod 23 within the wound of a patient while the balloon 16 of the bleeding control device 10 is inflated (as shown in FIGS. 32A and 32B), the rod 23 may be removed. The rod 23 is withdrawn from the housing 34 of the bleeding control device 10 and a valve (left side of FIG. 33) or gasket 36 seals the interior of the inflatable balloon 16 from the environment to prevent deflation of the inflatable balloon 16. Removal of the rod 23 prevents the possibility of a rod 23 within the patient being inadvertently moved which could cause damage or pain for the patient.

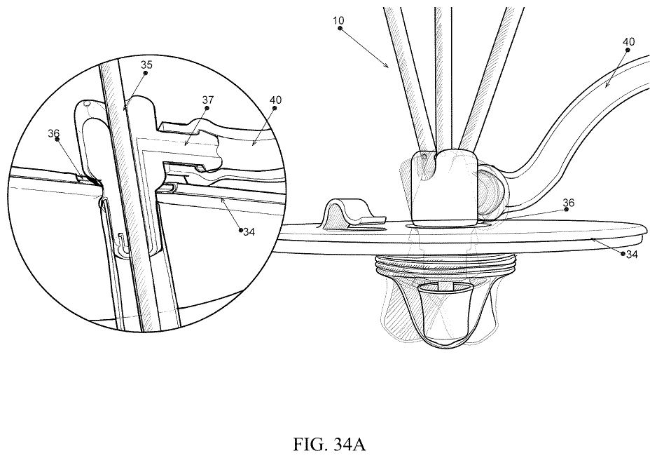

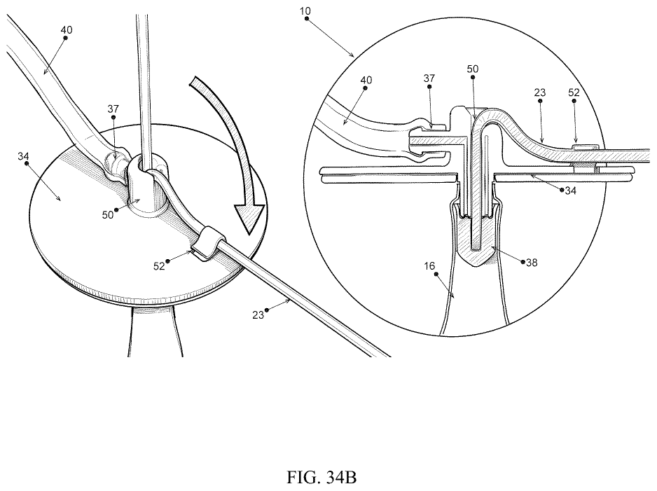

Referring to FIG. 34A, a bleeding control device 10 embodiment is shown. The bleeding control device 10 includes a plate-like shaped housing 34 that defines a central opening 35 with a gasket 36 arranged therein to seal against a rod 23. The housing 34 includes a fluid connector 37 that connects to tubing 40 for providing of an inflation medium to the housing 34. A rod 23 is partially inserted through the housing 34 and into the balloon 16 that is retained by the housing 34 (right side of FIG. 34A). The rod 23 is further inserted to navigate the balloon 16 into the wound of a patient as discussed above in connection with other embodiments.

Referring to FIG. 34B, after the navigation and inflation of the balloon 16, the rod 23 is withdrawn and bent over to manage the exposed portion 39 of the rod. A nub 50 on the proximal side of the housing is arranged to help keep the rod 23 bent, e.g. at 90°. A clip 52 is provided on the proximal side of the housing 34 to clip the exposed portion 39 of the rod 23 to keep the rod 23 laying flat and near the patient. The tip 38 of the rod 23 when pulled up, seats against a port on the distal side of the housing 34 to form a seal, thereby preventing inflation fluid in the inflatable balloon 16 from escaping back through the housing 34. Advantageously, in this state, the bleeding control device 10 is in a low profile for transport of the patient, e.g. from a location where the injury occurred to an ambulance, and then from the ambulance to a hospital room.

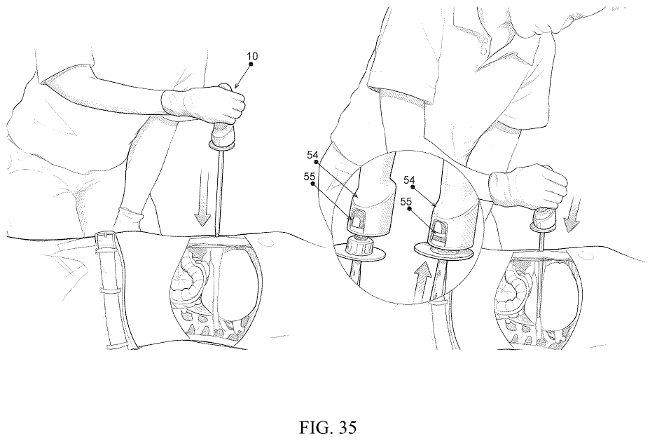

Referring to FIG. 35, a method of navigating a bleeding control device 10 in a patient is shown. A user (e.g. an EMT) carefully introduces the tip of the balloon 16 and catheter (or rod) 23 into the wound on the exterior of the body. The user carefully navigates the wound track with the catheter 23 while paying attention to the amount of force applied on a force gauge (or strain gauge) 54 (shown in enlargement bubble). The force gauge 54 is configured to indicate (at indicator 55) an amount of force being applied to the patient by the catheter.

Advantageously, the present disclosure provides bleeding control devices that provide new, optimized and efficient approaches to covering and blocking a wound to control bleeding.

Although various features have been shown in different figures for simplicity, it should be readily apparent to one of skill in the art that various features may be combined without departing from the scope of the present disclosure.

The foregoing description of embodiments of the present invention has been presented for the purpose of illustration and description. It is not intended to be exhaustive or to limit the invention to the form disclosed. Obvious modifications and variations are possible in light of the above disclosure. The embodiments described were chosen to best illustrate the principles of the invention and practical applications thereof to enable one of ordinary skill in the art to utilize the invention in various embodiments and with various modifications as suited to the particular use contemplated.

What is claimed is:

1. A method of controlling bleeding comprising:
providing a bleeding control device, the bleeding control device comprising:
a housing that defines a tool opening; and
an inflatable balloon held by the housing;
inserting a tool through the tool opening and into the inflatable balloon; and
inflating the inflatable balloon such that the inflatable balloon inflates within a wound of a patient, thereby mitigating bleeding from the wound.

2. The method according to claim 1, wherein the inserting the tool through the tool opening occurs prior to the inflating the inflatable balloon.

3. The method according to claim 1, wherein the wound is a bullet wound.

4. The method according to claim 1, wherein the wound is a stabbing wound.

5. A bleeding control device comprising
a housing that defines the tool opening; and
an inflatable balloon held by the housing;
wherein the housing and the tool opening are configured such that a tool can be inserted through the tool opening from a first side of the housing to a second side of the housing and into the inflatable balloon.

6. The bleeding control device according to claim 5, wherein the housing includes an inflation port and a fluid path way from the inflation port to an interior of the inflatable balloon.

7. The bleeding control device according to claim 6, further comprising a one-way valve arranged within the fluid pathway.

8. The bleeding control device according to claim 5, further comprising a sealing element arranged within the tool opening and configured to seal the tool while the tool is inserted through the tool opening.

9. The bleeding control device according to claim 5, further comprising an attachment element arranged at or on the second side of the housing.

10. The bleeding control device according to claim 9, wherein the attachment element is an adhesive.

11. The bleeding control device according to claim 9, wherein the attachment element is a sticker.

12. The bleeding control device according to claim 9, wherein the attachment element is a hooked surface.

13. The bleeding control device according to claim 5, wherein the second side of the housing has a substantially flat surface.

14. A bleeding control system comprising:
a tool; and
a bleeding control device comprising:
a housing that defines the tool opening; and
an inflatable balloon held by the housing;
wherein the housing and the tool opening are configured such that the tool can be inserted through the tool opening from a first side of the housing to a second side of the housing and into the inflatable balloon.

15. The bleeding control system according to claim 14, wherein the tool includes a break line configured such that the tool can be folded and/or broken along the break line.

16. The bleeding control system according to claim 14, wherein the tool includes one or more break lines along a longitudinal length of the tool each configured such that the tool can be folded and/or broken along the respective break line.

17. A bleeding control device comprising
a housing that defines the tool opening; and
an inflatable balloon held by the tool;
wherein the housing and the tool opening are configured such that a tool can be inserted through the tool opening from a first side of the housing to a second side of the housing and into the inflatable balloon.
